(19) United States
(12) Patent Application Publication
(10) Pub. No.: US 2022/0193379 A1
(43) Pub. Date: Jun. 23, 2022

(54) Floppy Distal End Section with Stiff Solid Shaft

(71) Applicant: Bard Access Systems, Inc., Salt Lake City, UT (US)

(72) Inventor: Glade H. Howell, Draper, UT (US)

(21) Appl. No.: 17/553,609

(22) Filed: Dec. 16, 2021

Related U.S. Application Data
(60) Provisional application No. 63/127,024, filed on Dec. 17, 2020.

(51) Int. Cl. A61M 25/09; A61M 25/06

(52) U.S. Cl. CPC A61M 25/09; A61M 25/065; A61M 2210/12; A61M 2025/09175; A61M 2025/09083

(57) ABSTRACT

A section is disclosed for use with a portion assembly. The section can include a solid core section with a distal section having a first diameter, and a proximal section having a second diameter, wherein the second diameter can be greater than the first diameter. The section can include a coil wound around the distal section of the solid core section. The proximal section can have a flexural stiffness that is less than a flexural stiffness of the distal section. A length of the distal section of the section may be shorter than the length of the portion. A length of the proximal section of the section may be longer than the length of the portion.

### PRIORITY

This application claims the benefit of priority to U.S. Provisional Application No. 63/127,024, filed Dec. 17, 2020, which is incorporated by reference in its entirety into this application.

### BACKGROUND

Portions, such as central venous portions are typically formed of a material having a relatively low durometer, which results in the portion having a lack of column strength. Column strength is typically advantageous during insertion and especially when a distal tip of the portion is displaced through the skin puncture site and the vascular puncture site. Column strength is also advantageous during further advancement of the portion through the puncture sites. In such an instance, column strength along the portion portion outside the vasculature is helpful while column strength along the portion within the vasculature can be detrimental to the guidance of the portion along the vascular path. In some cases, the portion may incorporate an introducer needle to establish the column strength for insertion of the portion. In such cases, the introducer needle may need to be removed prior to insertion of a section. In many cases, the section, while having stiffness and flexibility properties for guidance of the portion through the vasculature, lacks the column strength to facilitate advancement of the portion through the skin and vascular puncture sites. In some cases, both guiding capability and column strength for the portion are needed at the same time. As such, there is a need to provide column strength to the portion for advancement through the puncture sites while also providing guidance of the portion through the vascular path.

Disclosed herein are sections, portion assemblies, and methods that address the foregoing.

### SUMMARY

Disclosed herein is a section, including a distal section having a distal portion of a solid core section and a proximal section having a proximal portion of the solid core section. The distal section includes a coil wound around the solid core section, and the proximal section does not include a coil wound around the solid core section.

In some embodiments, the proximal section of the section has a flexural stiffness that is less than the distal section of the section. In some embodiments, a diameter of the proximal portion of the solid core section is greater than a diameter of the distal portion of the solid core section.

The distal section is configured for insertion of the portion into a vasculature of a patient and for guidance of the portion along the vasculature. The proximal section is configured to be inserted through a portion. The proximal section may also be configured to provide a column strength to the portion.

The section may be configured so that, during insertion, the proximal portion provides a column strength to a proximal portion of the portion while the distal portion simultaneously provides guidance to a distal portion of the portion along a vasculature.

The proximal portion of the solid core section may define a stiffness of the section along the proximal section and may also define an outside diameter of the section along the proximal section. In some embodiments, the proximal section is longer than the distal section. In some embodiments, the section includes an atraumatic distal tip.

Disclosed herein is a portion assembly including a portion and a section. The portion comprises a soft portion tube and a lumen extending along a length of the portion, wherein the length extends from a proximal end to a distal end of the portion. The lumen also extends through the soft portion tube. The section comprises a distal section including a distal portion of a solid core section, and a proximal section including a proximal portion of the solid core section, wherein a diameter of the solid core section along the proximal portion is greater than a diameter of the solid core section along the distal portion.

In some embodiments, a length of the distal section is shorter than the length of the portion, and in some embodiments, a length of the proximal section is longer than the length of the portion. A distal end of the section may be disposed within the lumen.

During use, the distal section of the section may extend distally beyond the distal end of the portion, and the proximal section of the section may be disposed through the lumen of the portion.

In some embodiments, the portion assembly includes an introducer portion disposed through the lumen of the portion, and the introducer portion includes an introducer portion lumen. The introducer needle may be disposed at least partially through the introducer portion lumen. In some embodiments, the introducer needle is disposed only through a distal portion of the introducer portion lumen, and the section is disposed through a proximal portion of the introducer portion lumen.

Disclosed herein is a method of using a section comprising the following steps. The method includes a step of obtaining a section, wherein the section includes a distal section including a distal portion of a solid core section, and a proximal section including a proximal portion of the solid core section, wherein a diameter of the solid core section along the proximal portion is greater than a diameter of the solid core section along the distal portion. The method includes a step of obtaining a portion including a lumen extending a length of the portion from a distal end to a proximal end of the portion. The method includes a step of threading the section through the lumen of the portion and into a vasculature of a patient, while the portion is disposed external to the patient. The method includes a step of positioning the section within the lumen so that the proximal section is disposed within the lumen from the distal end to the proximal end of the portion. The section is also positioned so that a distal end of the proximal section is positioned adjacent a distal tip of the portion. The method includes a step of inserting the distal tip of the portion together with the distal end of the proximal section through a vascular puncture site. The method includes a step of advancing the portion through the vasculature along the section.

In some embodiments, the advancing step includes adjusting the position of the section within the lumen so that the distal end of the proximal section is positioned adjacent the vascular puncture site. The advancing step may further include displacing the portion together with the section distally into the vasculature a first distance, and retracting proximally the section through the lumen the first distance so that the distal end of the proximal section is repositioned adjacent the vasculature puncture site. The advancing step may further include repeating the displacing and retracting steps.

In some embodiments, the inserting step includes applying a distally-directed compression force to the portion and the section while simultaneously grasping the portion at a proximal end and grasping the proximal portion of the section.

In some embodiments, the portion includes an introducer needle disposed within the lumen of the portion, and the method includes a step of removing the introducer needle from the lumen prior to the threading step.

These and other features of the concepts provided herein will become more apparent to those of skill in the art in view of the accompanying drawings and following description, which describe particular embodiments of such concepts in greater detail.

### DRAWINGS

FIG. 1A is a side view of a section, in accordance with some embodiments.

FIG. 1B is a cross-section detail view of a portion of the section of FIG. 1A, in accordance with some embodiments.

FIG. 2 illustrates a portion assembly including the section of FIG. 1A, in accordance with some embodiments.

FIG. 3A illustrates the portion assembly of FIG. 2 in a first state of insertion into a patient, in accordance with some embodiments.

FIG. 3B illustrates the portion assembly of FIG. 2 in a second state of insertion into a patient, in accordance with some embodiments.

FIG. 3C is detailed cross-sectional illustration of a portion of the portion assembly of FIG. 3B, in accordance with some embodiments.

FIG. 3D illustrates the portion assembly of FIG. 2 in a third state of insertion into a patient, in accordance with some embodiments.

FIG. 3E is detailed cross-sectional illustration of a portion of the portion assembly of FIG. 3D, in accordance with some embodiments.

### DETAILED DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or a “proximal-end portion” of, for example, a portion disclosed herein includes a portion of the portion intended to be near a clinician when the portion is used on a patient. Likewise, a “proximal length” of, for example, the portion includes a length of the portion intended to be near the clinician when the portion is used on the patient. A “proximal end” of, for example, the portion includes an end of the portion intended to be near the clinician when the portion is used on the patient. The proximal portion, the proximal-end portion, or the proximal length of the portion can include the proximal end of the portion; however, the proximal portion, the proximal-end portion, or the proximal length of the portion need not include the proximal end of the portion. That is, unless context suggests otherwise, the proximal portion, the proximal-end portion, or the proximal length of the portion is not a terminal portion or terminal length of the portion.

With respect to “distal,” a “distal portion” or a “distal-end portion” of, for example, a portion disclosed herein includes a portion of the portion intended to be near or in a patient when the portion is used on the patient. Likewise, a “distal length” of, for example, the portion includes a length of the portion intended to be near or in the patient when the portion is used on the patient. A “distal end” of, for example, the portion includes an end of the portion intended to be near or in the patient when the portion is used on the patient. The distal portion, the distal-end portion, or the distal length of the portion can include the distal end of the portion; however, the distal portion, the distal-end portion, or the distal length of the portion need not include the distal end of the portion. That is, unless context suggests otherwise, the distal portion, the distal-end portion, or the distal length of the portion is not a terminal portion or terminal length of the portion.

Unless defined otherwise, all technical and scientific terms used herein have the same meaning as commonly understood by those of ordinary skill in the art.

As set forth above, there is a need to provide column strength to a proximal portion of the portion for advancement of the portion through the puncture sites and a simultaneous need to provide guidance of a distal portion of the portion along the vascular path. Disclosed herein are sections, portion assemblies, and methods that address the foregoing.

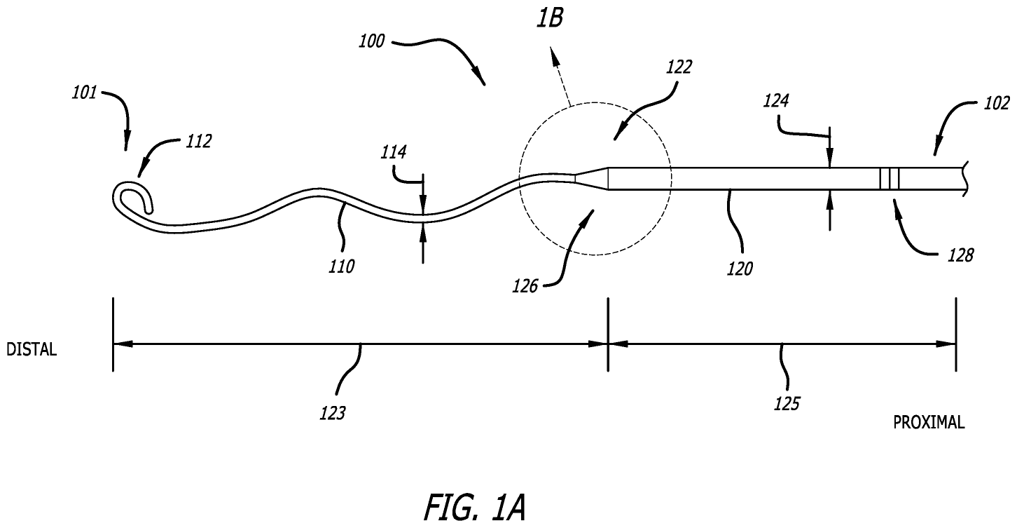

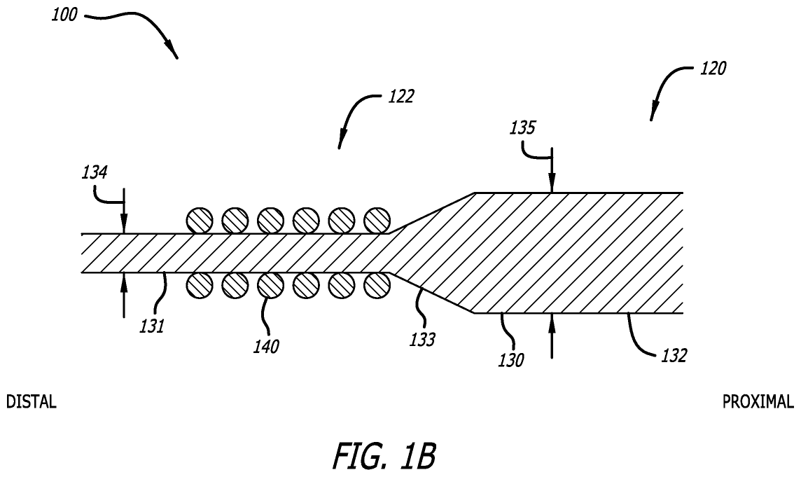

FIG. 1A is a side view of a section 100, in accordance with some embodiments described herein. The section 100 may be used in conjunction with a portion as described below. The section 100 may be configured to facilitate insertion of a portion into a vasculature of a patient. The section 100 includes a distal section 110 extending to a distal end 101, and a proximal section 120 extending to a proximal end 102.

The distal section 110 may be configured to be disposed within a vasculature of a patient. In some embodiments, the distal section 101 may comprise a flexibility that is consistent with medical sections configured to traverse a vasculature. As such, the distal section 110 may comprise a flexibility sufficient to traverse a vasculature without causing injury to the vascular wall. In other words, the distal section 110 may flex during insertion to conform with the vasculature structure without plastic deformation, kinking, and/or breaking. The distal section 110 may also comprise sufficient stiffness to facilitate insertion via a distally applied compression force without buckling within the vasculature. In some embodiments, the distal section 110 may include a length sufficient to extend from the vasculature insertion site to a desired location within the vasculature such as the lower ⅓ of the superior vena cava (“SVC”) of the heart, for example. As such, placement of intravascular devices may include use of the section 100 as an intravascular section. The distal section 110 may comprise a round cross section having a diameter 111 consistent with insertion through the portion and the vasculature.

In some embodiments, the distal section 110 may comprise a preformed shape 112 to aid insertion through the vasculature such as an atraumatic tip. In some embodiments, the preformed shape 112 may aid in preventing injury or damage to the soft tissues of the vasculature during advancement and/or retraction of the section 100. In some embodiments, the preformed shape 112 includes a curved portion adjacent the distal tip 101. The curved portion may be configured so that a distal extension of a longitudinal axis of the section intersects the section. Such an embodiment may cause a curved portion of the section to contact the vascular wall and thereby inhibit direct contact of the distal tip 101 with the vascular wall. The distal section 110 includes a length 123.

The proximal section 120 may be configured to be manually inserted into a lumen of a portion. The proximal section 120 may be configured for manual handling by a clinician and may comprise a flexural stiffness that facilitates manual displacement of the proximal section 120 distally into the portion without plastic deformation, kinking, and/or breaking. The stiffness of the proximal section 120 may enhance a stiffness of the portion, i.e., provide a column strength to the portion to aid insertion of the portion into the patient. The proximal section 120 may comprise a diameter 124 consistent with disposition of the proximal section 120 within the portion. In some embodiments, the diameter 124 may be the same, larger, or smaller than a diameter 114 of the distal section 110. In some embodiments, the proximal section 120 may be configured to be at least partially disposed within the vasculature of a patient. In some embodiments, a length 125 of the proximal section 120 may be longer than the length 123 of the distal section 110.

In some embodiments, the proximal section 120 may include indicia 128. The indicia 110 may be indicative of a distance to a distal end 126 of the proximal section 120. In some instances, the distal end of the portion may not be visible to a clinician, such as an instance where the distal end is disposed within the patient. A location of indicia 128 with respect to a proximal end of the portion may indicate a position of the distal end 126 of the proximal section 120 with respect to the distal end of the portion. The indicia 110 may also be indicative of a distance to the distal end 101 of the section 100. In some instances, a clinician may observe the indicia 128 to assess the position of the distal end 101 along the vasculature of the patient.

The proximal section 120 may comprise a distal transition portion 122. The distal transition portion 122 may define a smooth transition of physical properties between the distal section 110 and the proximal section 120. The distal transition portion 122 may comprise a taper to transition the diameter 114 of the distal section 110 to the diameter 124 of the proximal section 120. The distal transition portion 122 may also be constructed to transition the flexibility of the distal section 110 to the stiffness of the proximal section 120. In some embodiments, the distal transition portion 126 may define a strain relief.

FIG. 1B illustrates an embodiment of a structure of the section 100. The section 100 may be constructed of a solid core section 130 extending a length of the section 100. In some embodiments, the section 130 may be formed of a nitinol material. The section 130 includes a distal section portion 131 and a proximal section portion 132 that correspond with the distal and proximal sections 110, 120, respectively. The distal section portion 131 may include a thin diameter 134 consistent with the flexibility of the distal section 110. The proximal portion 132 may include a thick diameter 135 consistent with the stiffness of the proximal section 120. In the illustrated embodiment, the stiffness of the proximal section 120 may be defined by a flexural stiffness of the proximal portion 132 of the section 130. Flexural stiffness is understood as the product of the elastic modulus (E) of a material and the area moment of inertia (I) where the flexural stiffness (EI) has the SI units of Newtons (N)·meters² (m²) or N·m². In the case of a solid circular cross-section, (I) is defined by the following equation 1 where (d) is the diameter of the circular cross-section. In the illustrated embodiment, (d) may be the thick diameter 135 of the proximal portion 132 of the section 130.

$$I = \frac{π}{64} {d}^{4} \qquad Equation 1$$

The section 130 may include a section transition portion 133. The section transition portion 133 may include a taper between the thin diameter 134 and the thick diameter 135. The section transition portion 133 may inhibit plastic deformation, kinking, and/or breaking of the section 130 at the interface between the thin diameter 134 and the thick diameter 135. The section transition portion 133 may define a strain relief of the section 130. The distal section portion 131 may be wound with a coil 140. A diameter of the coil 140 may define the diameter 114 of the distal section 110, and the thick diameter 135 of the section 130 may define the diameter 124 of the proximal section 120. In some embodiments, the thick diameter 135 of the section 130 may define the stiffness of the proximal section 130.

The section may include alternative structures of the section 100. For example, the section 130 may comprise a constant diameter, such as the diameter 134, along the length of the section 100. The proximal portion 132 of the section may be include a cannula disposed around and attached to the section 130 to define the diameter 124 of the proximal section 120 and to add stiffness to the section 130. Other alternative structures, as may be contemplated by one of ordinary skill, are also considered part of this disclosure.

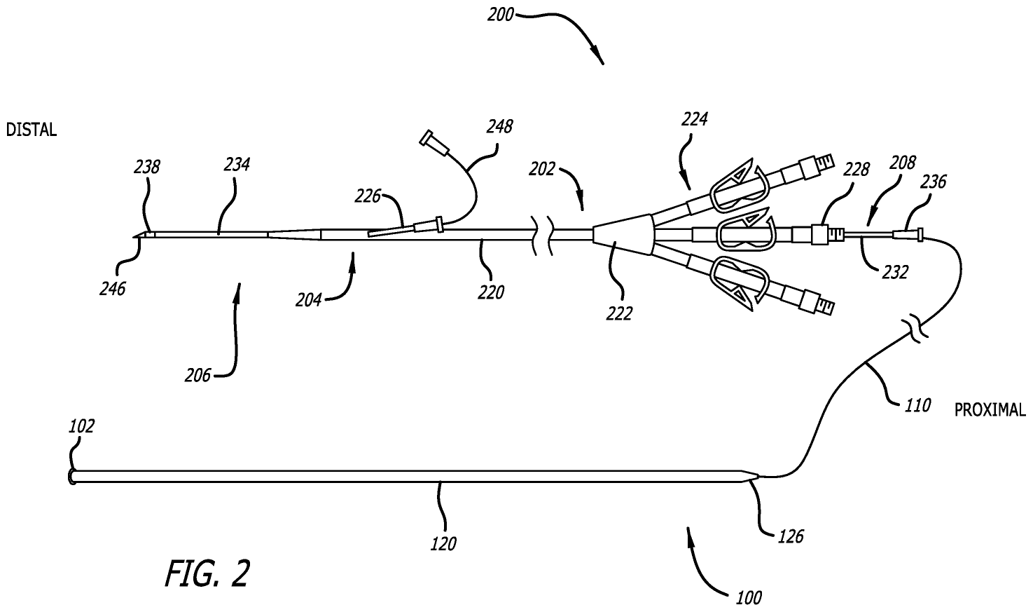

FIG. 2 illustrates a portion assembly 200 including the section 100 of FIG. 1A. In some embodiments, FIG. 2 illustrates the portion assembly 200 in a ready-to-deploy state. As shown, the portion assembly 200 includes a portion 202 and an introducer 206 coupled together. The portion 202 may be a monoluminal or multiluminal portion including at least a primary lumen 208 extending through the portion 202. The portion 202 includes a soft portion tube 220, a portion hub 222, and one or more extension legs 224 corresponding with one or more lumens extending at least partially through the soft portion tube 220. In the illustrated embodiment, the primary lumen 208 corresponds with a primary leg 228. The portion tube 220 includes a distal tip 230 about a distal-end portion of the soft portion tube 220 corresponding to the distal end of the portion 202.

The introducer 206 includes an introducer portion 232 and an introducer needle 226. The introducer portion 232 includes an introducer-portion tube 234, an introducer-portion hub 236 about a proximal-end portion of the portion tube 234, and a distal tip 238 at a distal end of the introducer-portion tube 234. The introducer 206 is coupled to the portion 202, such that the introducer-portion tube 234 is disposed within the primary lumen 208. When coupled together, the introducer-portion tube 234 extends 1) distally beyond the distal tip 230 of the portion and 2) proximally beyond a proximal end of the primary leg 228.

In some embodiments, the portion 202 includes a side aperture 204 extending through a wall of the soft portion tube 220 into the primary lumen 208 and further through a wall of the introducer-portion tube 234 into a single lumen of the introducer portion 232. The introducer needle 226 is disposed through the side aperture 104 and into the single lumen of the introducer portion 232 such that a beveled tip 246 of the introducer needle 126 extends past the distal tip 238 of the introducer portion 232 for establishing a percutaneous puncture. In some embodiments, the side aperture 204 may be omitted and the introducer needle 226 may be inserted into the introducer portion 232 via the introducer-portion hub 236.

In some embodiments, the portion assembly 100 may further include an access section 248 that may be disposed in a needle lumen of the introducer needle 126 such that a distal end of the access section 248 is proximal the beveled tip 246 of the introducer needle 226. This allows for immediate advancement of the distal end of the access section 248 beyond the beveled tip 246 of introducer needle 226 and into the vasculature upon establishing access thereto. In other embodiments, the access section 248 may be omitted.

As stated above, the portion assembly 200 includes the section 100 in accordance with some embodiments. In the illustrated embodiment, the section 100 may be disposed in the single lumen of the introducer portion 232 such the distal end 101 of the section 100 is proximal of the side aperture 204 but distal of the portion hub 222, which allows for immediate advancement of the distal end 101 of the section 100 into the vasculature upon removing the introducer needle 226 from the lumen of the introducer portion 232. In some embodiments, the proximal section 120 of the section 100 may be disposed proximal of the introducer-portion hub 236. In some embodiments, a portion of the distal section 110 of the section 100 may also be disposed proximal of the introducer-portion hub 236. In other embodiments, a portion of the proximal section 120 may disposed within the single lumen of the introducer portion 232. In some embodiments, the section 100 may not be inserted into the single lumen of the introducer portion 232 when the portion assembly 200 is in the ready-to-deploy state.

In some embodiments, the length 125 of proximal section 120 may be sufficiently long to extend from a proximal end of the portion to a distal end of the portion. In some embodiments, the length 125 may be sufficiently long so that, when the distal end 126 of the proximal section 120 is disposed adjacent the distal end of the portion, a portion of the proximal section 120 extends proximally away from the proximal end of the portion. The extending portion of the proximal section 120 may be sufficiently long such that the clinician may grasp and manipulate the proximal section 120.

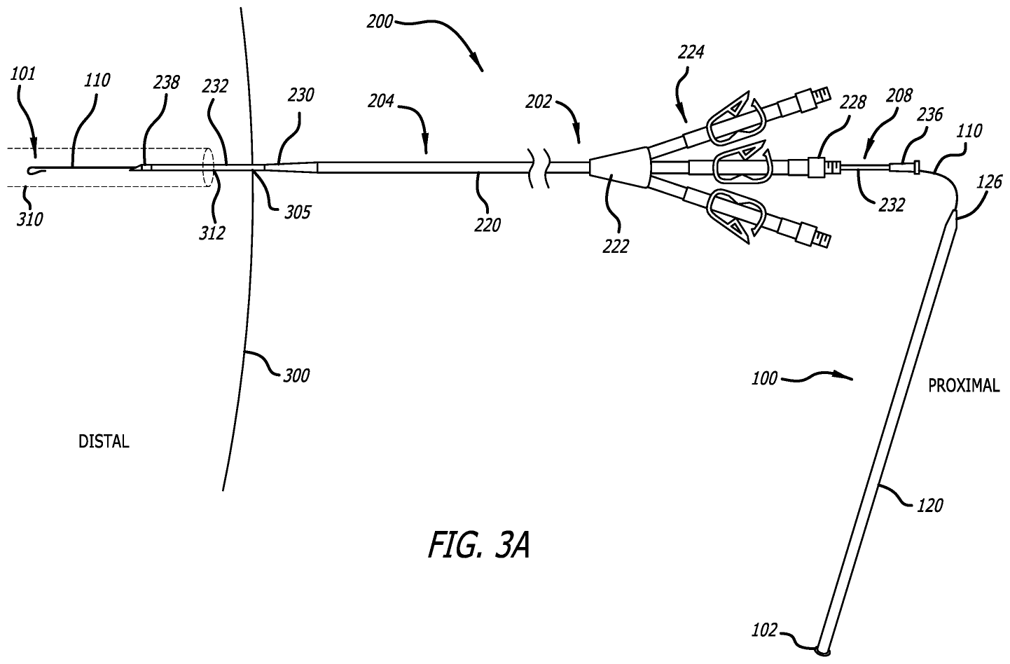

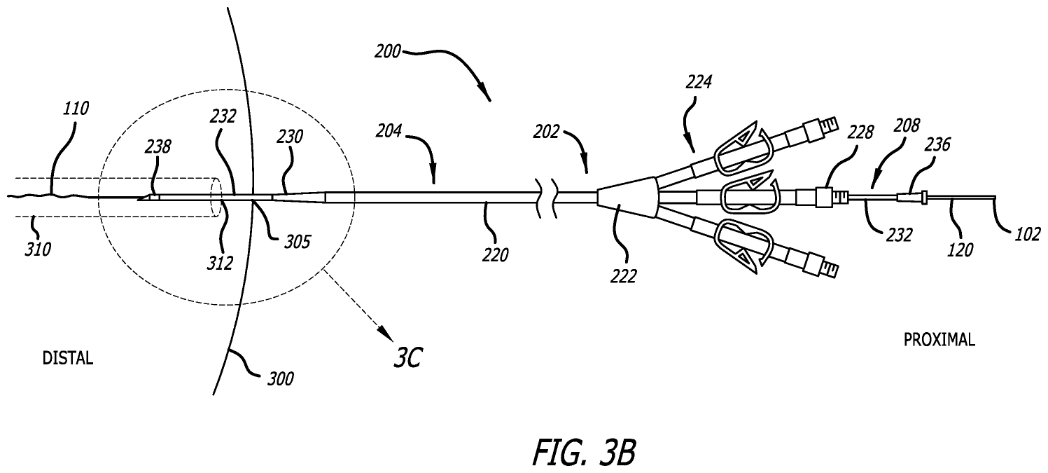

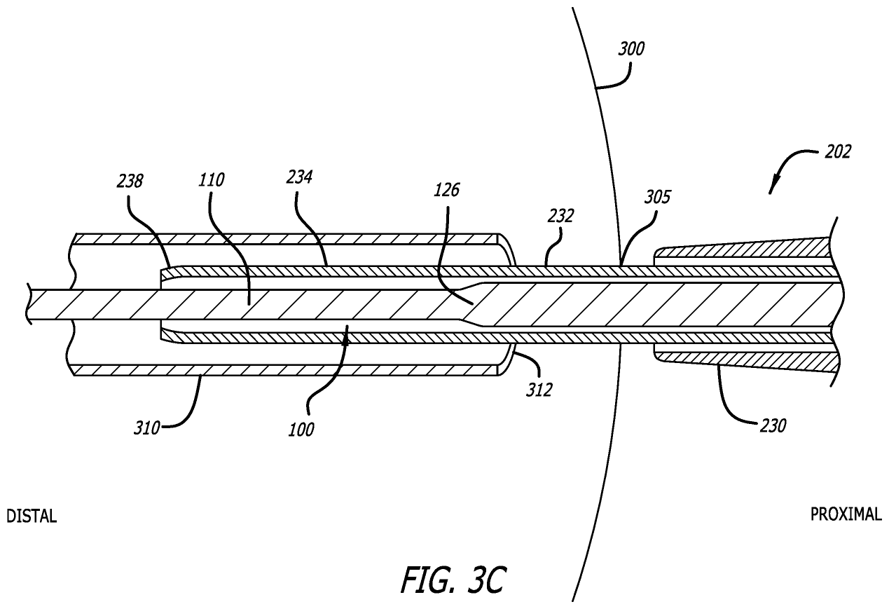

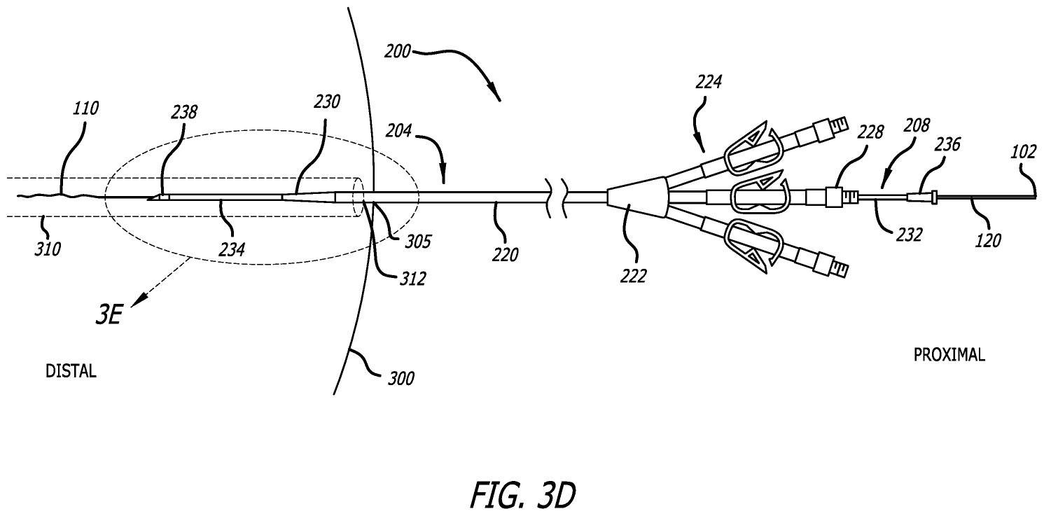

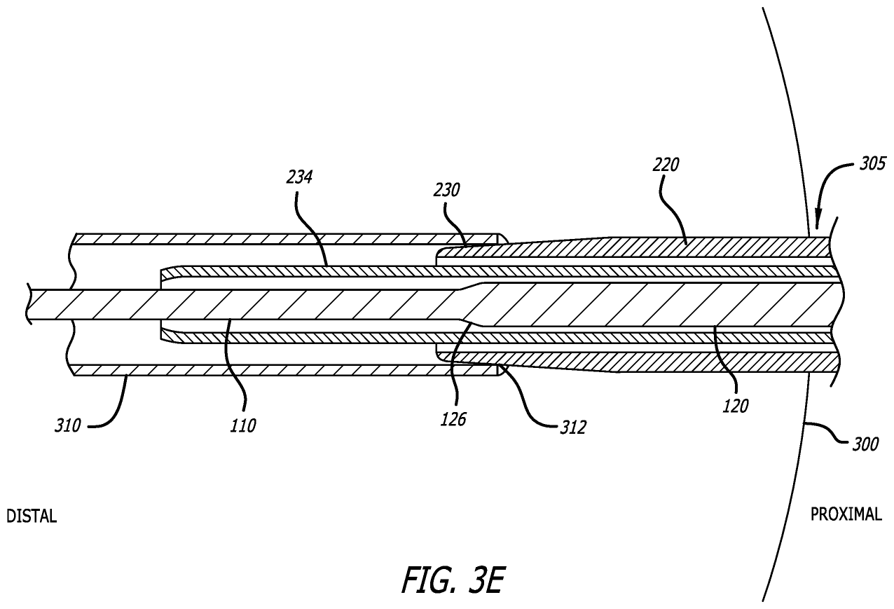

FIGS. 3A, 3B and 3D illustrate the portion assembly 200 in a first, a second, and a third state of insertion into a patient, respectively, in accordance with some embodiments. FIG. 3A illustrates the portion assembly 200 in a first state of insertion where the introducer needle 226 has punctured the skin of a patient 300 at a skin puncture site 305. The introducer needle 226 has also punctured the blood vessel 310 at the blood vessel puncture site 312 to establish a needle tract. The introducing portion 232 is also inserted along with the introducer needle 226 so that the distal tip 238 of the introducing portion 232 is disposed within the blood vessel 310. The introducer needle 226 and the access section 248 have been withdrawn from the introducing portion 232 and the side aperture 204. The section 100 has been distally advanced so that a distal end 101 of the section 100 extends distally beyond the distal tip 238 and into the blood vessel 310.

FIG. 3B illustrates a second state of insertion wherein the portion 202 remains at the same position as illustrated in FIG. 3A. With reference to both FIGS. 3B-3C, the section 100 has been distally advanced so that the distal end 126 of the proximal section 120 is disposed within the introducing-portion tube 234 at a location adjacent the blood vessel puncture site 312. The distal section 110 is disposed within the blood vessel 310. The proximal section 120 of the section 100 is disposed along an entire length of the portion 202. In other words, the proximal section 120 extends proximally from the blood vessel puncture site 312 beyond a proximal end of the portion 202. As the proximal section 120 of the section 100 is disposed along the entire length of the portion 202, the stiffness of the section provides added stiffness, i.e., column strength, to the portion 202 to facilitate insertion of the portion tube 220 into the blood vessel 310. In some instances, the distal tip 230 of the portion 202 may cause expansion of the skin puncture site 305 and the blood vessel puncture site 312. In such instances, the force required to insert the portion 202 may be consistent with the enhanced stiffness provided by the proximal section 120.

Referring specifically to FIG. 3C, a detailed cross-sectional view of the distal portion of the portion 202 and the introducer portion 232 at the skin and blood vessel puncture sites, 305, 312 when the portion is in the second state of insertion is shown. The introducing-portion tube 234 is shown disposed through the skin puncture site 305 and blood vessel puncture site 312. The distal tip 230 of the portion 202 remains proximal the skin and blood vessel puncture sites, 305, 312. The proximal section 120 of the section 100 is shown extending into the blood vessel 310 so that the distal portion 126 of the proximal section 120 is disposed a small distance distal the blood vessel puncture site 312. In some instances, when the distal portion 126 is adjacent (in close proximity to) the blood vessel puncture site 312, the distal end 101 of the section 100 may be within the SVC and therefore, further distal advancement of the section 100 may be detrimental to the patient.

FIG. 3D illustrates a third state of insertion wherein the portion 202 has been inserted into the blood vessel 310 so that the distal tip 230 of the portion 202 is inserted into the blood vessel 310. In this third state, the skin puncture site 305 and the blood vessel puncture site 312 may have been expanded by the tapered portion of the distal tip 230 during insertion. The proximal section 120 disposed along the portion 202 may have provided a column strength to facilitate insertion of the distal tip 230 and expansion of the skin puncture site 305 and the blood vessel puncture site 312 by the distal tip 230.

FIG. 3E is a detailed cross-sectional view of the distal portion of the portion 202 and the introducer portion 232 at the skin and blood vessel puncture sites, 305, 312 when the portion 202 is in the third state of insertion. The section 100 has been retracted proximally so that the distal portion 126 of the proximal section 120 is positioned adjacent (in close proximity to) the blood vessel puncture site 312. Accordingly, the proximal section 120 extending proximally from the introducer-portion hub 236 is greater in length in the third insertion state as illustrated in FIG. 3C than in the second insertion state as illustrated in FIG. 3B.

In some embodiments, the process of inserting the portion 202 as illustrated in FIGS. 3B and 3D may be repeated multiple times during insertion of the portion 202. In other words, the steps of 1) inserting the portion a distance and 2) proximally retracting the section 100 relative to the portion 202 a corresponding distance, so that the distal end 126 remains in adjacent the blood vessel puncture site 312, may be repeated one or more times as the portion 202 is inserted to its intended final position.

### Methods

Any methods disclosed herein include one or more steps or actions for performing the described method. The method steps and/or actions may be interchanged with one another. In other words, unless a specific order of steps or actions is required for proper operation of the embodiment, the order and/or use of specific steps and/or actions may be modified. Moreover, sub-routines or only a portion of a method described herein may be a separate method within the scope of this disclosure. Stated otherwise, some methods may include only a portion of the steps described in a more detailed method.

Methods of using the section 100 may include the following steps or processes. A method may include a step of inserting the section 100 into the lumen 208 of a portion 202 before the portion 202 is inserted into the patient. In some embodiments, the distal end 101 of the section 100 is disposed within the lumen 208. The section may enter the lumen 208 at a proximal end of the lumen 208. In some embodiments, a distal portion of the distal section 110 of the section 100 may be disposed within the lumen 208 while a proximal portion of the distal section 110 is disposed outside the lumen 208. In other embodiments, the entire distal section 110 may be disposed within the lumen 208. In some embodiments, a distal portion of the proximal section 120 may be disposed within the lumen. In some embodiments, the section 100 into the lumen 208 of a portion 202 after the portion 202 is at least partially inserted into the patient

In some embodiments, the portion may include an introducer portion 232, including an introducer-portion lumen, disposed within the lumen 208. In such embodiments, the inserting step may include inserting the section 100 into the introducer-portion lumen. In such embodiments, the distal end 101 of the section 100 is disposed within the introducer-portion lumen. The section may enter the introducer-portion lumen at a proximal end of the introducer-portion lumen. In some embodiments, a distal portion of the distal section 110 of the section 100 may be disposed within the introducer-portion lumen while a proximal portion of the distal section 110 is disposed outside the introducer-portion lumen. In other embodiments, the entire distal section 110 may be disposed within the introducer-portion lumen. In some embodiments, a distal portion of the proximal section 120 may be disposed within the introducer-portion lumen.

A method may further include a step of inserting the introducing section 248 into the introducer-portion lumen. The introducing section 248 may be inserted through a side aperture 204 entering into the introducer-portion lumen. The introducing section 248 may be further inserted distally through the introducer-portion lumen and positioned so that a distal end of the introducing section 248 is positioned within the introducer-portion lumen between the side aperture 204 and the distal tip 238 of the introducer portion 232.

A method may further include a step of establishing a needle tract from an area of skin 300 to the blood vessel 310 of the patient with the beveled tip 246 of the introducer needle 226. In some embodiments, the method may include advancing the introducer needle 226 through the side aperture 204. As set forth above with respect to the ready-to-deploy state of the portion assembly 200 illustrated in FIG. 3A, the introducer needle 226 may extend through the side aperture 204 of the soft portion tube 220 of the portion 202, extend through the side of the introducer-portion tube 234 of the introducer portion 232, and exit out of the distal tip 238 of the introducer portion 232.

A method may further include a step of advancing the access section 248 through the introducer needle 226 and into the blood vessel 310 of the patient.

A method may further include a step of advancing the distal-end portion of the introducer portion 232 into the blood vessel 310 over the access section 248.

A method may further include a step of withdrawing the introducer needle 226 from both the blood vessel 310 and the portion assembly 200 leaving the access section 248 and the introducer portion 232 inserted within the blood vessel 310.

A method may further include a step of advancing the section 100 through the introducer portion 232 and into the blood vessel 310. In some embodiments, the step of advancing the section 100 may include first withdrawing the introducer needle 226 from both the blood vessel 310, the lumen 208 and the introducer portion 232.

A method may further include a step of advancing the section 100 through the introducer portion 232 such that the stiff proximal section 120 of the section 100 is disposed within the portion 202 thereby adding stiffness to the portion 202.

A method may further include a step of positioning the section 100 within the portion 202 such that a distal end 126 of the proximal section 120 of the section 100 is disposed adjacent the distal tip 230 of the portion 202.

A method may further include a step of positioning the section 100 within the portion 202 such that a distal end 126 of the stiff proximal section 120 of the section 100 is disposed adjacent the blood vessel puncture site 312.

A method may further include a step of advancing both the introducer portion 232 and the portion 202 farther into the blood-vessel 310 a first distance while simultaneously grasping the proximal section 120 of the section 100 and the portion 202.

A method may further include a step of proximally retracting the section 100 the first distance within the portion 202 such that the distal end 126 of the proximal section 120 of the section 100 is repositioned adjacent the blood vessel puncture site 312.

A method may further include a step of advancing the portion 202 through the vasculature along the section 100.

A method may further include a step of removing the section 100 from the portion 202.

A method may further include a step of removing the introducing portion 232 from the portion 202.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

What is claimed is:

1. A section, comprising:
a solid core section including a proximal section and a distal section; and
a coil wound around the distal section of the solid core section, wherein the proximal section has a flexural stiffness that is less than a flexural stiffness of the distal section.

2. The section of claim 1, wherein a diameter of the proximal portion of the solid core section is greater than a diameter of the distal portion of the solid core section.

3. The section of claim 1, wherein the distal section is configured for insertion into a patient vasculature.

4. The section of claim 1, wherein the distal section is configured to guide a portion along the patient vasculature.

5. The section of claim 1, wherein the proximal section is configured to be inserted through a portion, and wherein the proximal section provides a column strength to the portion.

6. The section of claim 1, wherein the section is configured so that, during insertion, the proximal section provides a column strength to a proximal portion of the portion while the distal section simultaneously provides guidance to a distal portion of the portion along the patient vasculature.

7. The section of claim 1, wherein the flexural stiffness of the proximal section is configured to enable manual displacement of the proximal section into the portion without kinking or breaking.

8. The section of claim 1, wherein the proximal portion of the solid core section defines an outer diameter of a portion of the section.

9. The section of claim 1, wherein the proximal section is longer than the distal section.

10. The section of claim 1, further comprising an atraumatic distal tip.

11. A portion assembly comprising:
a portion; and
a section disposed at least partially through a lumen of the portion, the section including a solid core section and comprising:
a distal section having a first diameter, and
a proximal section having a second diameter,
wherein the second diameter is greater than the first diameter.

12. The portion assembly of claim 11, wherein a length of the distal section of the section is shorter than the length of the portion.

13. The portion assembly of claim 11, wherein a length of the proximal section of the section is longer than the length of the portion.

14. The portion assembly of claim 11, wherein a distal end of the section is disposed within the lumen.

15. The portion assembly of claim 11, wherein, during use, the distal section of the section extends distally beyond the distal end of the portion, and wherein the proximal section of the section is disposed through the lumen.

16. The portion assembly of claim 11, further comprising:
an introducer portion disposed through the lumen, the introducer portion including an introducer portion lumen; and
an introducer needle disposed at least partially through the introducer portion lumen.

17. The portion assembly of claim 16, wherein the introducer needle is disposed only through a distal portion of the introducer portion lumen, and wherein the section is disposed through a proximal portion of the introducer portion lumen.

18. A method of using a section comprising the steps of:
obtaining a section including a solid core section and comprising:
a distal section having a first diameter, and
a proximal section having a second diameter,
wherein the second diameter is greater than the first diameter;
obtaining a portion;
threading the section through the lumen and into a vasculature of a patient, wherein the portion is external to the patient;
positioning the section within a lumen of the portion so that the proximal section is disposed within the lumen, wherein a distal end of a proximal section is positioned adjacent a distal tip of the portion;
inserting the distal tip of the portion together with the distal end of the proximal section of the section through a vascular puncture site; and
advancing the portion through the vasculature along the section.

19. The method of claim 18, wherein the advancing step comprises adjusting the position of the section within the lumen so that the distal end of the proximal section is positioned adjacent the vascular puncture site.

20. The method of claim 18, wherein the advancing step further comprises:
displacing the portion together with the section distally into the vasculature a first distance; and
retracting proximally the section through the lumen the first distance so that the distal end of the proximal section is repositioned adjacent the vasculature puncture site.

21. The method of claim 20, wherein the advancing step further comprises repeating the displacing and retracting steps.

22. The method of claim 18, wherein the inserting step comprises applying a distally-directed compression force to the portion and the section while simultaneously grasping the portion at a proximal end and grasping the proximal portion of the section.

23. The method of claim 18, wherein the portion comprises an introducer needle disposed with the lumen of the portion,

24. The method of claim 18, wherein the method of claim 18 further comprises removing the introducer needle from the lumen prior to the threading step.
